(19) United States
(12) Patent Application Publication
(10) Pub. No.: US 2017/0135902 A1
(43) Pub. Date: May 18, 2017

(54) ALL-IN-ONE SYSTEM TO STORE AND TRANSPORT TEMPERATURE SENSITIVE ITEMS: DEVICES AND METHODS FOR CONTROLLING AND MONITORING TEMPERATURE AS WELL AS SECURITY IN REAL-TIME.

(71) Applicant: Stephen J. Scully, JR., Rochester, MN (US)

(72) Inventor: Stephen J. Scully, JR., Rochester, MN (US)

(21) Appl. No.: 15/332,478

(22) Filed: Oct. 24, 2016

Related U.S. Application Data
(60) Provisional application No. 62/245,316, filed on Oct. 23, 2015.

(51) Int. Cl. A61J 1/18; F25D 29/00; F25D 31/00; A61B 50/30; F25B 21/04

(52) U.S. Cl. CPC A61J 1/18; A61B 50/30; F25B 21/04; F25D 31/005; F25D 31/006; F25D 29/008; A61J 2200/42; A61J 2200/44; A61J 2200/50; A61J 2200/72; A61B 2050/0014

(57) ABSTRACT

This document provides devices and methods for the monitoring and maintenance of the temperature and the security of many medical reservoirs in a secure fashion that enables for safe storage and transport of medical reservoirs that can allow for easy documentation of a chain of custody and temperature history profile. For example, this document provides devices and methods for monitoring the temperature of blood in phlebotomy tubes. In some embodiments of the devices and methods, the temperature of the contents of the medical reservoirs are regulated, modulated, and/or maintained by the devices provided herein.

### BACKGROUND

1. Technical Field

This document relates to devices and methods for housing, monitoring, and maintaining the temperature of many medical reservoirs while maintaining a secure chain of custody and log of sample information, contents and product access in a secure manner. For example, this document relates to devices and methods for monitoring the temperature of blood, cerebrospinal fluid, or bone marrow in tubes or vials, vaccines in ampoules or bottles, and/or biological pharmaceutical products as well the detection of the physical presence of the item, the temperature status of these items, and whether any of these items have been accessed, inserted into the device, or removed by a verified customer or user.

2. Background Information

The Center for Disease Control reports that $300 million dollars was wasted in 2014 due to the improper storage and regulation of temperature for vaccines. If these vaccines and other biomedical samples (such as bone marrow, cerebrospinal fluid, or blood) as well as biomedical products (such as biological or biosimilar pharmaceutical products) freeze or get too warm, they can be ruined or lose potency and efficacy. Consequently, rigorous control of these sample and products storage temperatures, and verification of the temperatures to which blood samples have been exposed is advantageous. In addition, having a more reliable and traceable chain of custody for security purposes to prevent tampering and/or the introduction of counterfeit products for these samples and products is advantageous. In addition, it is advantageous if an electronic log can be kept each time the sample or product has been accessed. It is also advantageous if all these separate features could be combined into a mobile cooler for convenience of all users: customers and couriers.

### SUMMARY

This document provides devices and methods for monitoring and maintaining the temperature of biomedical reservoirs which has the capability to maintain a chain of custody and ensure the security of a sample or product through communications in real-time. For example, this document provides devices and methods for monitoring and maintaining and regulating the temperature of vaccines in ampoules, vials, or bottle, while also tracking location and position of the product. In one example of use, the devices provided herein will track and maintain temperatures to prevent excess temperatures above or below 4 degrees Celsius while monitoring access of the reservoir.

In one implementation, this medical reservoir temperature monitoring and maintenance device includes tracking many smaller modular devices that include: a housing that defines structure for selective coupling to many smaller units that are composed of smaller devices that are individually tracking medical reservoirs; a user interface coupled to the housing; and a flexible temperature sensor that can be coupled to the housing. The temperature sensor is configured and operable to measure temperature of a materials that are within the unit when the medical reservoir and smaller devices are coupled to the housing. A pressure sensor may also be used to determine in the smaller items and devices have been removed to make authorized users that tampering is occurring. The user interface includes an alarm functionality for notifying a user that the temperature sensor detected the temperature of the material to be outside of a predetermined range of acceptable temperatures and also if the pressure from stretching of the housing occurs and its contact from the reservoir changes such as when or if the sample or product has been removed from the device.

Such a medical reservoir temperature monitoring and maintenance device may optionally include one or more of the following features. The structure defined by the housing for selective coupling to the medical reservoirs may be included by a stretchable elastic and/or conformable plastic that allows for the tube to slip through, or could wrap around a bottle. The structure defined by the housing for selective coupling to the medical reservoir may comprise thin and small elements that are flexible and can be enclosed by a stretchable plastic that is conformable and may stick to the bottle. The structure defined by the housing may be an open space or an adjustable rack, matrix, or array to accommodate different items that need to be stored at different temperatures. The medical reservoir may be a vaccine bottle or therapeutic ampoule. The temperature sensor may comprise a micro thermistor, or STT-MTJ sensor that have previously been described under US Patent Pub. #2012/0181651 A1. The user interface may include a display. The user interface may include one or more electrical buttons. The user interface may be available through a mobile device or a website. The medical reservoir temperature monitoring and maintenance device may further comprise an electrical connector coupled to the housing. The medical reservoir temperature monitoring and maintenance device may be configured and operable to wirelessly communicate with a separate computer or other device through cellular, satellite, wifi, or low energy Bluetooth methods. The medical reservoir temperature monitoring and maintenance device may be configured so that it can be resistant to sterilization procedures for reusability, or it could be disposable. The medical reservoir temperature monitoring and maintenance device may further comprise a heating and cooling device (such as a micro Peltier device, or newer classes of thermoelectric-cooling mechanisms based on the adiabatic spin entropy expansion) coupled to the housing. The cooling and heating device may be configured and operable to heat and cool the material within the medical reservoir when the medical reservoir is coupled to the housing. The data from the temperature sensor and pressure or stretch sensor may be periodically stored in memory that is coupled to the housing. That memory may also be periodically transmitted wirelessly by various methods.

In another implementation, a method for monitoring a temperature of contents of a medical reservoir includes coupling a medical reservoir temperature monitoring device to the medical reservoir; on a periodical timing interval basis, storing temperature values from the temperature sensor in memory that is coupled to the housing; and based on the temperature values, providing temperature information via the user interface or wirelessly to a separate unit that records this information on many units. The medical reservoir temperature monitoring device includes: a housing, the housing defining structure for selective coupling to a medical reservoir; a temperature sensor coupled to the housing, pressure and/or stretch sensors coupled to the housing, the temperature sensor being configured and operable to measure temperature of a material within the medical reservoir when the medical reservoir is coupled to the housing; the pressure or stretch sensor being configured and operable to measure the pressure of a material within the housing, when the medical reservoir is coupled to the housing and a user interface coupled to the housing or transmissible to a separate mobile unit, the user interface including an alarm functionality for notifying a user that the temperature sensor detected the temperature of the material to be outside of a predetermined range of acceptable temperatures as well as status of the battery of the device and whether the pressure or stretch sensor has detected a stimulus outside of a predetermined range, or has changed.

In another embodiment, the rack to which smaller units are attached may be gyroscopic, so that it moves to prevent samples being inverted. In this embodiment, the device would be aware of inversions that may occur, especially if the device is inverted too quickly. In addition, the device may be modular so that many of them can fit together on a palette for more convenient shipping by freight.

Such a method for monitoring a temperature and pressure of contents of a medical reservoir, or the stretching of the housing may optionally include one or more of the following features. The temperature, and/or pressure, and/or stretch information may be connected to a local or remote alarm. The pressure and or stretch information may comprise a local alarm. The time interval may be within a range that is less than 1 seconds or longer, up to 5 minutes. The method may further comprise transmitting the stored temperature and pressure or stretch values to an external computer or device. The transmitting may be performed wirelessly. The user interface may include a display, and temperature values and pressure and stretch values measured by the temperature sensor may be indicated on the display. The method may further comprise heating or cooling, by the medical reservoir temperature monitoring device, the material within the medical reservoir, wherein the heating or cooling is initiated based on the temperature values. The medical reservoir may be a phlebotomy tube.

Particular embodiments of the subject matter described in this document can be implemented to realize one or more of the following advantages. In some embodiments, the medical reservoir temperature monitoring and maintenance devices provided herein detect and store in memory a real-time and time-based record of the temperature and of the contents (e.g., blood) of a phlebotomy tube, and its presence or absence, attempts to remove the reservoir, and may insure that temperature deviations beyond normal body temperature 37 degrees Celsius are minimal if they occur. Using the temperature record, a clinical pathologist or laboratory healthcare worker can verify that the sample has not reached a temperature that could be detrimental to the sample and ultimately the patient if an incorrect diagnosis is made based on this type of pre-analytical error. In conclusion, patient safety and clinical results can be improved.

In some embodiments, the medical reservoir temperature monitoring and maintenance devices provided herein will provide a visual notification or silent alarm if the sample deviates from an acceptable temperature range. Such notifications or alarms can assist individuals to readily identify samples that may require rejection and an immediate request for a new sample(s) to be drawn, or for additional quarantine or further analysis of that suspect sample if needed or if necessary. In some embodiments, the temperature data will be available for download from the temperature monitoring device to another computing device. Historical data records can thereby be maintained in conjunction with an electronic medical record. In some embodiments, the temperature monitoring devices provided herein are portable and thereby allow portability of the medical reservoirs while continuing to monitor the temperature of the contents. In some embodiments, the medical reservoir temperature monitoring devices provided herein are reusable, and in particular embodiments the devices can be decontaminated by sterilization, and thus reused.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description herein. Other features, objects, and advantages of the invention will be apparent from the claims.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A modular device that is durable and can control and track temperature and security comprising:
a housing, the housing defining a gyroscopic tray or rack for smaller devices and medical reservoirs with selective coupling to these medical reservoirs;
multiple temperature sensors coupled to the housing, the temperature sensors configured and operable to measure temperature of many items within the device when smaller devices and/or medical reservoirs are placed inside the housing;
a stretch or pressure transducer that can detect when the bottle or vial of sample or product was placed in the device and if it has ever since changed position or location; and
a user display interface coupled to the housing, the user interface including an alarm functionality for notifying a user that the temperature sensor detected the temperature of the material to be outside of a predetermined range of acceptable temperatures and if the vial or sample has ever been removed.

2. The medical reservoir temperature monitoring device of claim 1, wherein the structure defined by the housing for selective coupling to the medical reservoir is box shaped structure with openings that can be accessed on top or on the side.

3. The medical reservoir temperature monitoring device of claim 1, wherein the structure defined by the housing for selective coupling to the medical reservoir includes vacuum insulated panels that use nanotechnology for a very high level of insulation.

4. The medical reservoir temperature monitoring device of claim 1, wherein the medical reservoir is a phlebotomy tube or therapeutic ampoule.

5. The medical reservoir temperature monitoring device of claim 1, wherein the temperature sensor comprises a thermistor.

6. The medical reservoir temperature monitoring device of claim 1, wherein the heating and cooling is provided by Adiabatic Spin-Entropy Expansion.

7. The medical reservoir temperature monitoring device of claim 1, wherein the user interface includes one or more electrical buttons.

8. The medical reservoir temperature monitoring device of claim 1, further comprising an electrical connector coupled to the housing for downloading data or recharging the power.

9. The medical reservoir temperature monitoring device of claim 1, wherein the medical reservoir temperature monitoring device is configured and operable to wirelessly communicate with a separate computer, device, or can transmit data by WiFi, Bluetooth, cellular, or satellite.

10. The medical reservoir temperature monitoring device of claim 1, wherein the medical reservoir temperature monitoring device is configured to be water resistant and is able to be sterilized.

11. The medical reservoir temperature monitoring device of claim 1, further comprising a cooling and heating device coupled to the housing, the cooling/heating device being configured and operable to cool and or heat the material within the medical reservoirs when the medical reservoirs are coupled to the housing. This cooling and heating may be accomplished by use of a Peltier device configured by an H bridge.

12. The medical reservoir temperature monitoring device of claim 1, wherein data from the temperature sensor is periodically stored in memory that is coupled to the housing.

13. A method for monitoring a temperature of contents of a medical reservoir with a data log and chain of custody, the method comprising:
coupling a medical reservoir temperature monitoring device to the medical reservoir, wherein the medical reservoir temperature monitoring device comprises:
a housing, the housing defining structure for selective coupling to a medical reservoir;
a pressure transducer or stretch sensor that can detect if there has been removal of the vial or bottle of the sample or product;
a temperature sensor coupled to the housing, the temperature sensor being configured and operable to measure temperature of a material within the medical reservoir when the medical reservoir is coupled to the housing; and
a user interface coupled to the housing, the user interface including an alarm functionality for notifying a user that the temperature sensor detected the temperature of the material to be outside of a predetermined range of acceptable temperatures;
on a periodic time interval basis, storing temperature values from the temperature sensor in memory that is coupled to the housing; and
based on the temperature values, providing temperature information via the user interface.

14. The method of claim 13, wherein the temperature information can comprise an alarm.

15. The method of claim 13, wherein the stretch or change in pressure information can comprise an alarm.

16. The method of claim 13, wherein the time interval is within a range between about 100 microseconds and 5 minutes.

17. The method of claim 13, further comprising transmitting the stored temperature values and other sensors of smaller devices within the device to an internal or external memory device.

18. The method of claim 17, wherein the transmitting is performed wirelessly.

19. The method of claim 13, wherein the user interface includes a display and temperature values measured by the temperature sensor are indicated on the display.

20. The method of claim 13, further comprising heating or cooling, by the medical reservoir temperature monitoring device, the material within the medical reservoir, wherein the heating or cooling is initiated based on the temperature values.
